(19) United States
(12) Patent Application Publication
(10) Pub. No.: US 2004/0122076 A1
(43) Pub. Date: Jun. 24, 2004

(54) Sulphonamides for the treatment of central nervous system diseases

(76) Inventors: Frank-Gerhard Bobb, Wuppertal (DE); Ulf Bruggemeier, Leichlingen (DE); Stephan-Nicolas Wirtz, Wuppertal (DE)

Correspondence Address: Jeffrey M Greenman, Bayer Corporation, 400 Morgan Lane, West Haven, CT, 06516, US

(21) Appl. No.: 10/415,455

(22) PCT Filed: Oct. 19, 2001
(86) PCT No.: PCT/EP01/12098

(30) Foreign Application Priority Data
Oct. 30, 2000 (DE) 100 53 813.4

(51) Int. Cl. A61K 31/405; A61K 31/4035

(52) U.S. Cl. 514/415; 514/416

(57) ABSTRACT

The present invention relates to the use of sulphonamides for the production of a medicament for the prophylaxis and/or treatment of diseases which are treatable using a 5-HT₆ antagonist, in particular of diseases of the central nervous system.

The present invention relates to the use of sulphonamides for the production of a medicament for the prophylaxis and/or treatment of diseases which are treatable using a 5-HT₆ antagonist, in particular of diseases of the central nervous system.

The phototechnical use of N-aryl-benzenesulphonamides is disclosed, for example, in U.S. Pat. No. 3,482,971 and/or U.S. Pat. No. 3,925,347.

WO 90/09787 discloses N-aryl-arenesulphonamides as radio- and chemosensitizing agents in cancer therapy.

EP-A-0 815 861 describes N-indolyl-benzenesulphonamides having affinity for the 5-HT₆ receptor for the control of central nervous system disorders.

N-Aryl-arenesulphonamides having 5-HT₆ receptor-antagonistic action for the treatment of diseases of the central nervous system are disclosed in WO 98/27081, WO 99/02502, WO 99/37623 and WO 00/12073.

The compounds according to the invention and their use as antiviral medicaments are the subject of International Patent Application No. PCT/EP 00/03492.

Surprisingly, it has been found that compounds described in the International Application No. PCT/EP 00/03492 exhibit 5-HT₆ receptor-antagonistic action and are therefore suitable for the prophylaxis and/or treatment of diseases which are treatable by antagonism of the 5-HT₆ receptor.

The invention therefore relates to the use of compounds of the general formula (I)

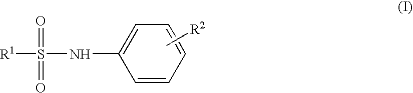

in which

R¹ represents a group which is selected from the following formulae

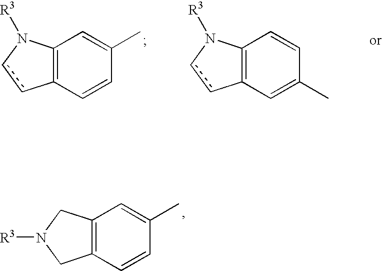

in which

[symbol] represents a single or a double bond,

R³ represents hydrogen, (C₁-C₆)alkyl or (C₃-C₆)cycloalkyl, which in each case can be substituted by 1 to 3 substituents which are selected from the group which consists of hydroxyl, halogen, amino, mono- or di(C₁-C₆)alkylamino, (C₁-C₆)alkanoylamino, (C₁-C₆)alkanoyloxy, (C₁-C₆)alkanoyl, carboxyl, (C₁-C₆)alkoxycarbonyl, carbamoyl, mono- or di(C₁-C₆)alkylaminocarbonyl and cyano, or

R³ represents (C₆-C₁₀)arylsulphonyl, (C₆-C₁₀)arylcarbonyl, whose (C₆-C₁₀)aryl group in each case can be substituted by 1 to 3 substituents which are selected from the group which consists of halogen, (C₁-C₃)alkyl, carboxyl, (C₁-C₃)alkoxycarbonyl, carbamoyl, mono- or di(C₁-C₆)alkylaminocarbonyl, cyano, hydroxyl and (C₁-C₃)alkoxy, or

R³ represents (C₁-C₆)alkanoyl, (C₁-C₆)alkylsulphonyl, (C₃-C₆)cyclo-alkylcarbonyl, camphorsulphonyl or (C₃-C₆)cycloalkylsulphonyl, or

R³ represents R⁴—X—CO— or R⁴—X—CS—, in which

X represents O, S, NR⁵, in which R⁵ represents hydrogen or (C₁-C₃)alkyl, and

R⁴ represents (C₁-C₆)alkyl, (C₃-C₆)cycloalkyl, (C₆-C₁₀)aryl or 5- to 10-membered heteroaryl, and

R² represents

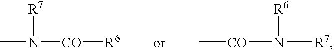

in which

R⁶ is (C₂-C₆)alkenyl or (C₁-C₈)alkyl, which is optionally mono- to trisubstituted identically or differently by amino, protected amino, (C₁-C₄)alkylamino, hydroxyl, cyano, halogen, azido, nitro, trifluoromethyl, carboxyl or phenyl, where phenyl for its part can be substituted up to two times, identically or differently, by nitro, halogen, hydroxyl, (C₁-C₄)alkyl or (C₁-C₄)alkoxy, or

R⁶ represents radicals of the formulae

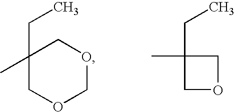

or -L-O—CO-Q,

in which

L represents a straight-chain or branched alkanediyl group having up to 6 carbon atoms,

Q represents (C₁-C₆)alkyl, which is optionally substituted by carboxyl, or

represents radicals of the formulae

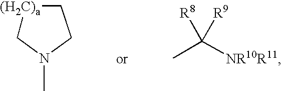

in which

a denotes the number 1 or 2,

R⁸ denotes hydrogen,

R⁹ denotes (C₃-C₈)cycloalkyl, (C₆-C₁₀)aryl or hydrogen, or denotes (C₁-C₈)alkyl,

where the (C₁-C₈)alkyl is optionally substituted by cyano, methylthio, hydroxyl, mercapto, guanidyl or by a group of the formula —N²R¹³ or R¹⁴—OC—,

in which

R¹² and R¹³ independently of one another denote hydrogen, (C₁-C₈)alkyl or phenyl,

and

R¹⁴ denotes hydroxyl, benzyloxy, (C₁-C₆)alkoxy or the abovementioned group —NR²R¹³,

or the (C₁-C₉)alkyl is optionally substituted by (C₃-C₈)cycloalkyl or by (C₆-C₁₀)aryl, which for its part is substituted by hydroxyl, halogen, nitro, (C₁-C₈)alkoxy or by the group —NR¹²R¹³,

in which R¹² and R¹³ have the meaning indicated above,

or the (C₁-C₈)alkyl is optionally substituted by, a 5- to 6-membered nitrogen-containing hetero-cycle or by indolyl, in which the corresponding —NH functions are optionally substituted by (C₁-C₆)alkyl or protected by an amino protective group,

R¹⁰ and R¹¹ are identical or different and denote hydrogen or an ammo protective group,

R⁷ represents hydrogen or a radical of the formula

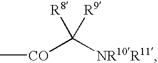

in which

R^{8′}, R^{9′}, R^{10′} and R^{11′} have the meaning of R⁸, R⁹, R¹⁰ and R¹¹ indicated above and are identical to or different from this,

and their salts

for the production of a medicament for the prophylaxis and/or treatment of diseases which are treatable using a 5-HT₆ receptor antagonist.

The substances according to the invention can also be present as salts. In the context of the invention, physiologically acceptable salts are preferred.

Physiologically acceptable salts can be salts of the compounds according to the invention with inorganic or organic acids. Preferred salts are those with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, lactic acid, benzoic acid, or methanesulphonic acid, ethanesulphonic acid, phenylsulphonic acid, toluenesulphonic acid or naphthalenedisulphonic acid.

Physiologically acceptable salts can likewise be metal or ammonium salts of the compounds according to the invention. Those particularly preferred are, for example, sodium, potassium, magnesium or calcium salts, and also ammonium salts which are derived from ammonia, or organic amines, such as, for example, ethylamine, di- or triethylamine, di- or triethanolamine, dicyclohexylamine, dimethylaminoethanol, arginine, lysine, ethylenediamine or 2-phenylethylamine.

The compounds of the general formula (I) according to the invention can occur in various stereochemical forms which either behave as image and mirror image (enantiomers), or which do not behave as image and mirror image (diastereomers). The invention relates both to the antipodes and to the racemic forms and the diastereomer mixtures. Just like the diastereomers, the racemic forms can be separated into the stereoisomerically uniform constituents in a known manner.

Certain compounds can furthermore be present in tautomeric forms. This is known to the person skilled in the art, and compounds of this type are likewise included by the scope of the invention.

(C₁-C₆)Alkyl in the context of the invention in general represents straight-chain or branched hydrocarbon radicals having 1 to 6 carbon atoms. Accordingly, (C₁-C₄)alkyl and (C₁-C₃)alkyl in the context of the invention in general represent straight-chain or branched-chain hydrocarbon radicals having 1 to 4, and 1 to 3 carbon atoms, respectively. Examples which may be mentioned are: methyl, ethyl, propyl, isopropyl, butyl, isobutyl, sec-butyl, tert-butyl, pentyl, isopentyl, hexyl and isohexyl.

(C₃-C₆)Cycloalkyl represents cycloalkyl groups having 3 to 6 carbon atoms [lacuna] includes, for example: cyclopropyl, cyclopentyl and cyclohexyl. Cyclopropyl is preferred.

The (C₁-C₆)alkoxy group, as is used in the present invention, and as is also used in the definitions of (C₁-C₆)alkoxycarbonyl, includes, for example, straight-chain or branched-chain alkoxy groups having 1 to 6 carbon atoms, particularly preferably alkoxy groups having 1 to 4 carbon atoms ((C₁-C₄)alkoxy), even more preferably alkoxy groups having 1 to 3 carbon atoms ((C₁-C₃)alkoxy). Examples which can be mentioned are methoxy, ethoxy, propoxy, isopropoxy, butoxy, isobutoxy, pentoxy, isopentoxy, hexoxy and isohexoxy. Preferred in methoxy, ethoxy and propoxy.

Mono- or di(C₁-C₆)alkylamino in the context of the invention includes those whose alkyl groups have 1 to 6 carbon atoms. These can be symmetrical or unsymmetrical alkylamino groups, such as, for example, dimethylamino, diethylamino, methylethylamino etc. This also applies to the mono- or di(C₁-C₆)alkylamino moiety in the mono- or di(C₁-C₆)alkylaminocarbonyl group.

(C₆-C₁₀)Aryl in the context of the invention represents an aromatic radical having 6 to 10 carbon atoms. Preferred aryl radicals are phenyl and naphthyl.

5- to 10-membered heteroaryl in the context of the invention represents 5- to 10-membered heteroatom-containing rings which can contain 1 to 8 heteroatoms in the ring, which are selected from O, S and N and include, for example, a pyridyl, furyl, thienyl, pyrrolyl, imidazolyl, oxazolyl, thiazolyl, isoxazolyl, triazolyl, thiadiazolyl, pyridazinyl, pyrazinyl, pyrimidinyl, indolicenyl, indolyl, benzo[b]thienyl, benzimdiazolyl, pyridoimidazolyl, indazolyl, quinolyl, isoquinolyl, naphthyridinyl, quinazolinyl, etc.

5- to 6-membered nitrogen-containing heterocycles include, for example: pyrrolidine, piperidine, piperazine, morpholine, pyridyl, furyl, thienyl, pyrolyl, imidazolyl, pyrazolyl, pyrazinyl, pyrimidinyl, pyridazinyl, etc.

Halogen in the context of the invention includes fluorine, chlorine, bromine and iodine. Preferred are chlorine or fluorine.

With respect to (C₆-C₁₀)arylsulphonyl and -carbonyl, reference may be made to the abovementioned definitions of (C₆-C₁₀)aryl.

(C₁-C₆)Alkanoyl, and (C₁-C₆)alkanoyl in the definition of (C₁-C₆)alkanoyloxy and (C₁-C₆)alkanoylamino, in the context of the invention represents straight-chain or branched-chain alkanoyl having 1 to 6 carbon atoms. Examples which may be mentioned are: formyl, acetyl, propanoyl, butanoyl, pentanoyl, pivaloyl and hexanoyl.

By the term “alkanediyl group having up to 6 carbon atoms”, straight-chain or branched-chain hydrocarbon groups are designated here which are linked to further radicals in two positions. Examples of alkanediyl groups are: —CH2-CH2-, —CH2-CH2-CH2-, —C(CH3)2—CH2-, —CH(CH3)-CH2-, —C(CH3)2-CH2-CH2-, CH(CH3)-CH2-CH2- etc.

Amino protective groups in the context of the invention are the customary amino protective groups used in peptide chemistry.

These preferably include: benzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyl-oxycarbonyl, 4-nitrobenzyloxycarbonyl, 2-nitrobenzyloxycarbonyl, 2-nitro-4,5-di-methoxybenzyloxycarbonyl, methoxycarbonyl, ethoxycarbonyl, propoxycarbonyl, isopropoxycarbonyl, butoxycarbonyl, isobutoxycarbonyl, tert-butoxycarbonyl, allyl-oxycarbonyl, vinyloxycarbonyl, 2-nitrobenzyloxycarbonyl, 3,4,5-trimethoxybenzyl-oxycarbonyl, cyclohexoxycarbonyl, 1,1-dimethylethoxycarbonyl, adamantylcarbonyl, phthaloyl, 2,2,2-trichloroethoxycarbonyl, 2,2,2-trichloro-tert-butoxycarbonyl, methyloxycarbonyl, phenoxycarbonyl, 4-nitrophenoxycarbonyl, fluorenyl-9-methoxycarbonyl, formyl, acetyl, propionyl, pivaloyl, 2-chloroacetyl, 2-bromoacetyl, 2,2,2-trifluoroacetyl, 2,2,2-trichloroacetyl, benzoyl, 4-chlorobenzoyl, 4-bromobenzoyl, 4-nitrobenzoyl, phthalimido, isovaleroyl or benzyloxymethylene, 4-nitrobenzyl, 2,4-dinitrobenzyl or 4-nitrophenyl.

5-HT₆ receptor antagonists within the meaning of the invention are compounds which bind to the human 5-HT₆ receptor and have an antagonistic action there. Preferably, these compounds exhibit in the assay described below, ‘binding to human recombinant 5-HT₆ receptors’, a K; of less than 10⁻⁵ M, and in one of the functional tests described below, ‘cAMP determinations’ and ‘h5HT₆ luciferase reporter gene test’, an antagonistic action (IC₅₀ value of less than 10⁻⁵ M).

Diseases which are treatable using a 5-HT₆ receptor antagonist are in particular diseases of the central nervous system. Examples which may be mentioned are cognitive disorders such as Alzheimer's disease, age-related memory disorders, dementia which occurs after stroke, frontotemporal dementia, vascular dementia, Korsakoff's syndrome and other forms of dementia (Sleight et al., Drug News and Perspectives 1997, 10, 214-224), Parkinson's disease, and also depression, schizophrenia and psychoses (Roth et al., J. Pharmacol. Exp. Ther. 1994, 268, 1403-1410). Likewise, the compounds according to the invention can be employed for the treatment of epilepsy (Routledge et al. Br. J. Pharmacol. 2000, 130, 1606-1612), migraine, anxiety, panic attacks, withdrawal symptoms, compulsive symptoms, sleep disorders, eating disorders (bulimia, anorexia), amyotrophic lateral sclerosis, multiple sclerosis, bone marrow injuries, Huntington's disease, craniocerebral trauma, Attention Deficit Hyperactivity Disorder (ADHD; WO 00/12073) and for the control of painful conditions.

Their use for the treatment of cognitive disorders, in particular Alzheimer's disease or other forms of dementia, is preferred.

In a preferred embodiment, the invention includes the use of compounds of the formulae

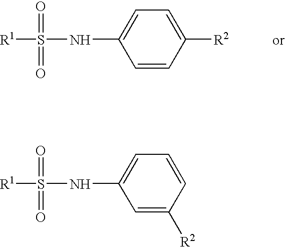

in which R¹ and R² have the meaning indicated above.

In a further preferred embodiment, the invention includes the use of compounds of the formulae

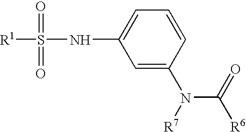

in which R¹, R⁶ and R⁷ have the meaning indicated above.

In a further preferred embodiment, the invention includes the use according to the invention of compounds of the general formula (I), in which:

R¹ represents a group which is selected from the formulae:

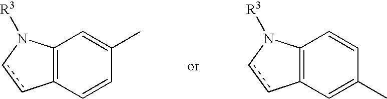

in which

[symbol] represents a single or a double bond, and

R³ has the meaning indicated above,

and their salts.

In a further preferred embodiment, the invention includes the use according to the invention of compounds of the general formula (I), in which:

R³ represents hydrogen, (C₁-C₆)alkyl or (C₁-C₆)alkanoyl, and

R² represents

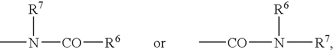

in which

R⁶ is (C₁-C₈)alkyl which is optionally substituted by halogen or hydroxyl, and

R⁷ is hydrogen,

and their salts.

In a particularly preferred embodiment, the invention includes the use according to the invention of compounds of the general formula (I) in which R⁶ is tert-butyl which is optionally substituted by halogen or hydroxyl, and their salts.

The compounds according to the invention can be prepared as follows:

In process (A), compounds of the general formula (II)

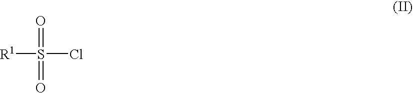

in which R¹ is as defined above, are reacted with compounds of the general formula (III)

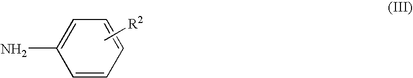

in which R² is as defined above, to give compounds of the general formula (I).

The reaction is preferably carried out in the presence of bases, such as pyridine, triethylamine and Hünig's base etc.

The reaction is preferably carried out in a solvent such as tetrahydrofuran, 1,4-dioxane, dichloromethane, etc.

The reaction is preferably carried out in a temperature range from −10° C. to 70° C.

The reaction is preferably carried out at normal pressure.

In process (B), compounds of the general formula (Ia):

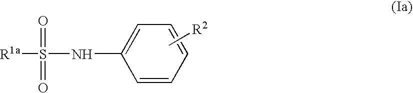

in which R² is as defined above, and

R^{1a} represents a group which is selected from the following formulae:

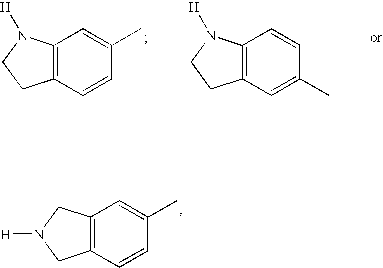

are reacted with compounds of the formula (IV):

R³-A (IV)

in which R³ is as defined above and A is a customary leaving group, in a manner known per se in the presence of a base to give compounds of the general formula (Ib):

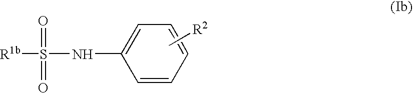

in which R² is as defined above and Rib represents a group which is selected from the following formulae:

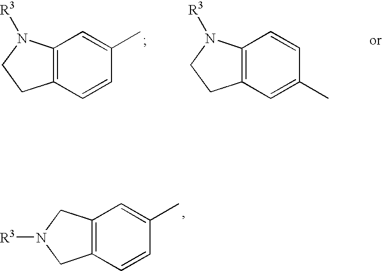

in which R³ is as defined above.

A in this case represents a customary leaving group used in nucleophilic substitution reactions, such as, for example, halogen (e.g. chlorine, bromine, iodine), OTs (Ts=tosyl) and OMes (Mes=mesyl).

Bases preferred in the reaction are tertiary amines, such as pyridine, Hünig's base etc., alkali metal hydroxide and alkali metal carbonate.

The reaction is preferably carried out in inert solvents such as tetrahydrofuran, 1,4-dioxane, dichloromethane, dimethylformamide etc.

The reaction is preferably carried out in a temperature range from −10° C. to 100° C.

The reaction is preferably carried out at normal pressure.

In process (C), compounds of the general formula (Ic)

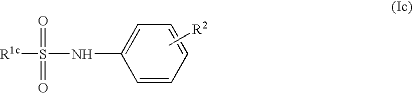

in which R² is as defined above and R^{1c} represents a group which is selected from the following formulae:

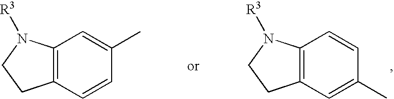

in which R³ is as defined above, are reacted by oxidation with DDQ (2,3-dichloro-5,6-dicyano-para-benzoquinone) in a manner known per se to give compounds of the general formula (Id):

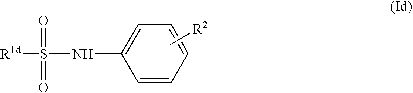

in which R² is as defined above, R^{1d} represents a group which is selected from the following formulae:

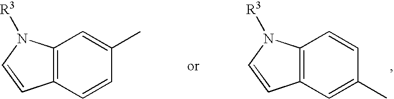

in which R³ is as defined above.

The reaction is preferably carried out in a solvent such as 1,4-dioxane or 1,2-dichloroethane.

The reaction is preferably carried out in a temperature range from room temperature up to the boiling point of the respective solvent at normal pressure.

The reaction is preferably carried out at normal pressure.

In process (D), compounds of the general formula (Ie)

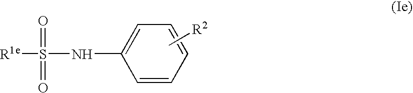

in which R² is as defined above and R^{1e} has the following formulae:

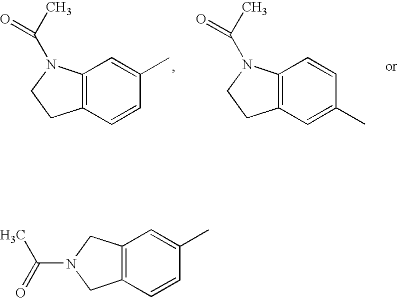

are reacted in a manner known per se in the presence of water with alkali metal hydroxides to give compounds of the formula (Ia).

Alkali metal hydroxides in this case include, for example, lithium hydroxide, sodium hydroxide, potassium hydroxide, etc, lithium hydroxide being preferred.

The reaction is preferably carried out in homogeneous aqueous solvent systems.

The reaction is preferably carried out in a temperature range from room temperature to 70° C.

The reaction is preferably carried out at normal pressure.

In process (E), compounds of the general formula (If)

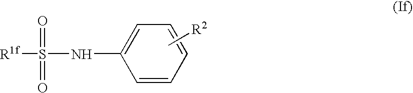

in which R² is as defined above and R^{1f} has the following formulae:

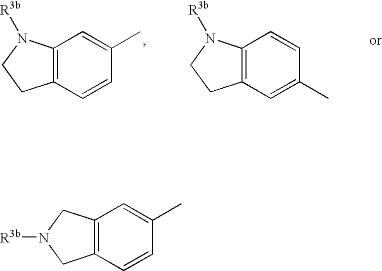

in which R^{3b} represents (C₁-C₆)alkanoyl, are reacted in a manner known per se with complex metal hydrides to give compounds of the general formula (Ig):

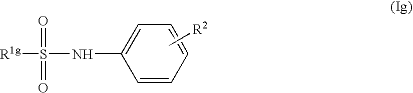

in which R² is as defined above and R^{1g} has the following formulae:

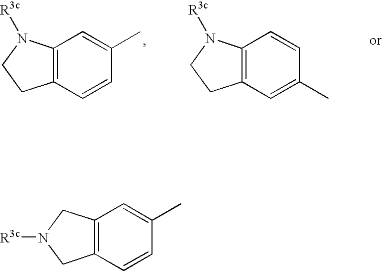

in which R^{3c} represents (C₁-C₆)alkyl.

Complex metal hydrides preferably used in the reaction are lithium aluminium hydride, diisobutylaluminium hydride, etc.

The reaction is preferably carried out in a solvent such as tetrahydrofuran, 1,4-dioxane etc.

The reaction is preferably carried out in a temperature range from −50° C. to 40° C.

The reaction is preferably carried out at normal pressure.

The processes according to the invention can be illustrated by the following reaction schemes.

The indole and indoline compounds can be prepared as follows:

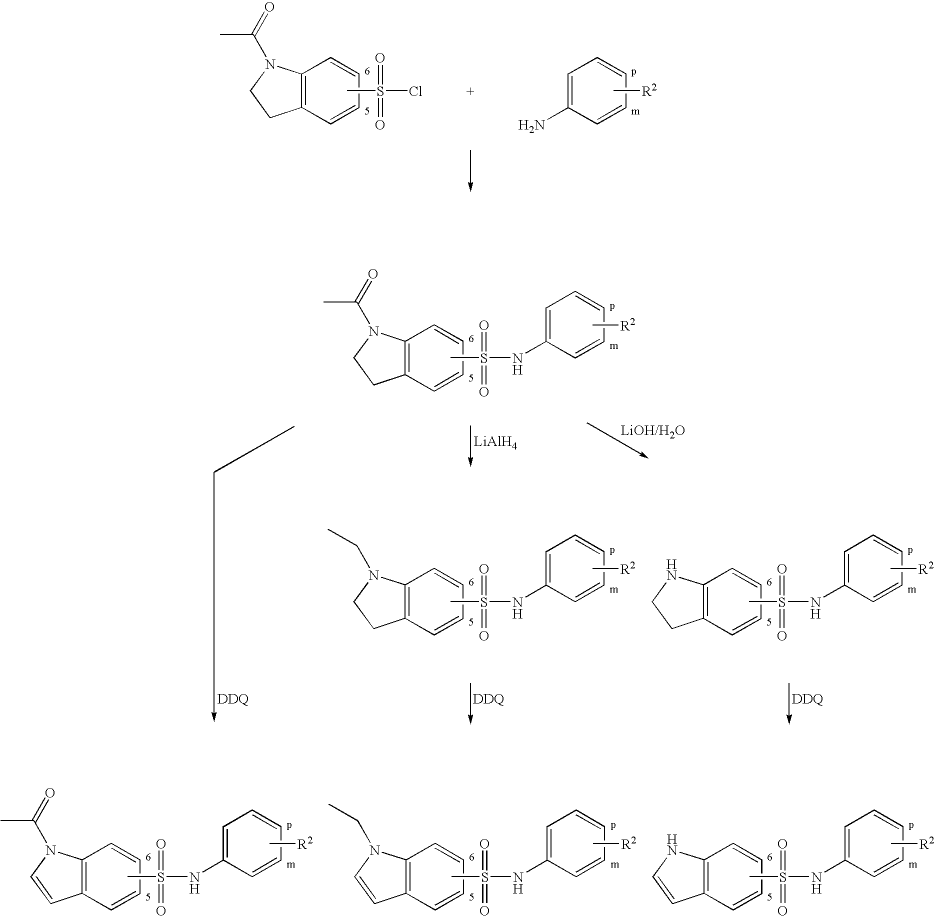

The compounds of the general formula (I) are then obtainable from the unsubstituted indole and indoline compounds (R³=hydrogen) by reaction with R³-A as described above.

The isoindoline compounds are obtainable, for example, according to the following scheme:

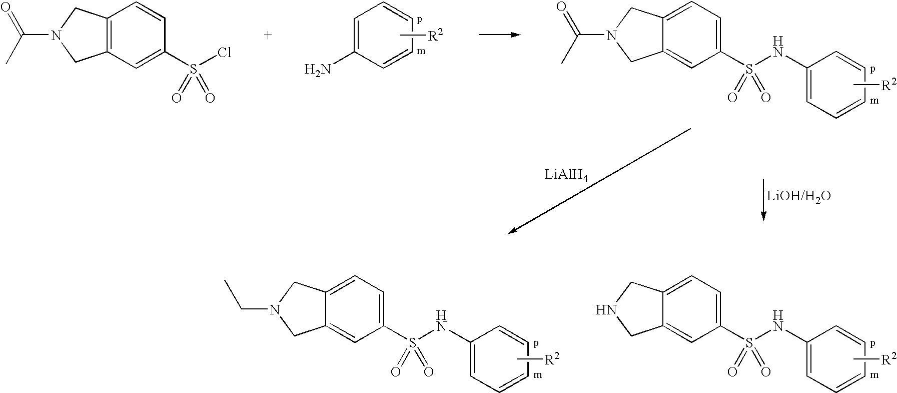

The compounds of the general formula (I) are then obtainable from the unsubstituted isoindoline compounds (R³=hydrogen) by reaction with R³-A as described above.

The preparation of the sulphonyl chloride starting compounds of the formula (II) is illustrated by the following reaction scheme:

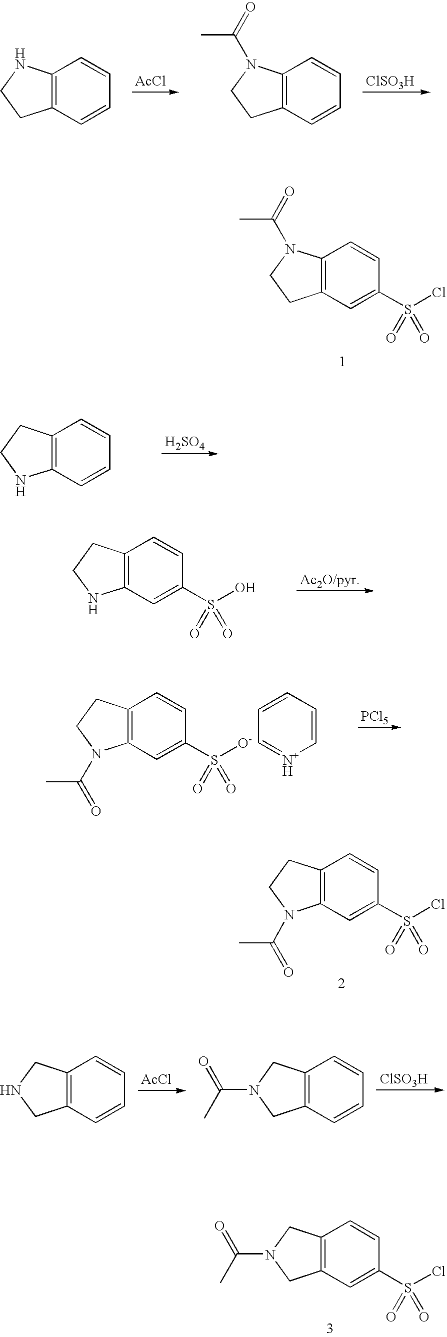

In this, the preparation of the sulphonyl chloride 1 is carried out, for example, according to A. L. Borrer, E. Chinoporos, M. Filosa, S. R. Herrchen, C. R. Petersen, C. A. Stern, J. Org. Chem. 53, 2047 (1988).

The sulphonyl chloride 3 can be prepared in analogy to the above reaction.

The preparation of the sulphonyl chloride 2 is carried out, for example, according to P. R. Carlier, M. P. Lockshin, M. P. Filosa, J. Org. Chem. 59, 3232 (1994).

The compounds of the general formula (I) are then obtainable from these compounds by reaction with the amines of the formula (Im), hydrolysis of the acetyl group, e.g. with LiOH/H₂O, and subsequent reaction with R³-A.

The preparation of the compounds of the general formula (III) is illustrated, for example, by means of the following reaction scheme:

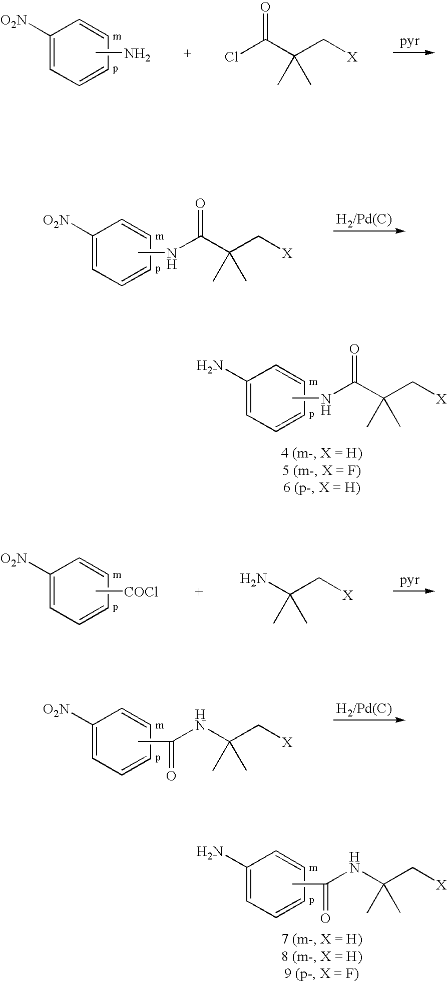

In this, pyr. denotes pyridine.

The aniline 4 is prepared, for example, according to U.S. Pat. No. 3,979,202.

The aniline 6 is prepared, for example, according to S. Rajappa, R. Sreenivasan, A. Khalwadekar, J. Chem. Res. Miniprint 5, 1657 (1986).

The aniline 7 is prepared, for example, according to WO 9631462.

The aniline 8 is prepared, for example, according to R. W. Hartmann, M. Reichert, S. Goehring, Eur. J. Med. Chem Chim. Ther. 29, 807 (1994).

The anilines 5 and 9 are prepared in an analogous manner.

With respect to the exact reaction conditions, reference may be made to the examples and starting examples.

Biological Tests

1. Binding to Human Recombinant 5-HT₆ Receptors

The binding of the compounds according to the invention to human recombinant 5-HT₆ receptors can be determined as follows. Membranes of HEK293 cells which express human recombinant 5-HT₆ receptors (RB-HS6, Receptor Biology, Inc., Beltsville Md. 20705, USA) were suspended in the ratio 1:40 in ice-cold sample buffer consisting of 50 mM tris HCl, 5 mM MgCl₂, 0.5 mM EDTA; 0.1% ascorbic acid and 10 μM pargyline (pH 7.4) and homogenized (Polytron). 100 μl of membrane suspension, 50 μl of [³H]-LSD (specific activity 75 Ci/mmol, final concentration 1 nNM) and 50 μl of test substance solution (8 different concentrations, 10⁻⁵ to 10⁻⁹ M) were incubated for 1 hour at 37° C. and the [³H]-LSD bound to the receptor was then separated from the free [³H]-LSD by filtration. The radioactivity retained by the filter was determined by scintillation spectroscopy. All tests were carried out in triplicate determinations. The IC₅₀ values were calculated using the program GraphPadPrism (Hill equation, special: one-site competition). The inhibition constant of the test substance Kᵢ was determined from the IC₅₀ value of the compounds (concentration of the test substance at which 50% of the ligand bound to the receptor is displaced), the dissociation constant K_{D} and the concentration L of [³H]-LSD (Kᵢ=IC₅₀/(1+L/K_{D})).

The compounds according to the invention have a Kᵢ of less than 10⁻⁵ M.

Example 44 has a Kᵢ value of 12 nM.

2. cAMP Determinations

The antagonistic action of 5-HT₆ ligands can be determined on HEK293 cells which express recombinant human 5-HT₆ receptors.

HEK293 cells which express recombinant human 5-HT₆ receptors are washed, detached from the culture dish, centrifuged twice and suspended again in Dulbecco's modified Eagle Medium (DMEM) without Phenol Red. 80 μl of suspension are transferred to a 96-hole plate at a density of 10 000 cells/hole and incubated at 37° C. for 30 min. Antagonists (10⁻⁹ to 10⁻⁴ M) are added in a volume of 20 μl/hole together with the agonist 5-HT (100 nM), pargyline (20 μM) and the phosphodiesterase inhibitor Ro 20-1724 (100 μM). After 20 min at 37° C., the incubation is ended by addition of 200 μl of ethanol and the samples are stored at −20° C. After centrifugation at 470 g (4° C., 5 min), 75 μl aliquots of the supernatant are transferred to Packard OptiPlates, evaporated in vacuo and taken up again in 0.05 m acetate buffer. The cAMP concentration is determined using the BIOTRAK cAMP [¹²⁵I] Scintillation Proximity Assay (SPA) System (Amersham). The concentration of the antagonist which results in 50% inhibition (IC₅₀), is calculated by means of the formula E=B+E₀*IC₅₀/(I+IC₅₀), where E is the measured cAMP concentration, E₀ is the concentration of cAMP produced in the presence of 100 nM 5-HT without antagonist, B is the basal value of the cAMP concentration and I is the concentration of the antagonist.

3. h5HT₆ Luciferase Reporter Gene Test

The 5-HT₆-agonistic action of compounds can be determined using this biological test.

Stock cultures of an HEK293-h5HT6 reporter cell line were prepared analogously to the method described in WO 98/37061, p. 55ff.

The following test protocol was used for substance screening: the stock cultures were grown under 5% CO₂ at 37° C. in -MEM containing 5% dialysed FCS and in each case split 1:10 after 3 days. Test cultures were inoculated into 96-hole plates at 5000 cells per well in Optimem Medium (GIBCO) and grown at 37° C. for 70 hours. The substances dissolved in DMSO were diluted 1× in medium and pipetted into the test cultures (maximum DMSO final concentration in the test batch: 0.5%). 10 minutes later, serotonin (5-HT) was added and the cultures were then incubated at 37° C. for 4 h in an incubator.

Activation of the 5-HT₆ receptors by 5-HT leads to the stimulation of adenylate cyclase and thus to the raising of the cAMP concentration in the cell. In the HEK293-h5HT6 luciferase system, the cAMP increase induces the expression of the reporter gene luciferase. Antagonists decrease this induction.

The supernatants were then removed and the cells were lysed by addition of 25 [lacuna] 1 of lysis reagent (25 mM triphosphate, pH 7.8 containing 2 mM DTT, 10% glycerol, 3% Triton X100). Directly thereafter, luciferase substrate solution (2.5 mM ATP, 0.5 mM luciferin, 0.1 mM coenzyme A, 10 mM tricine, 1.35 mM MgSO₄, 15 mM DTT, pH 7.8) was added, the mixture was briefly shaken, and the luciferase activity was measured using a Hamamatsu camera system.

The IC₅₀ values were calculated using the program GraphPadPrism (Hill equation, special: one-site competition).

The efficacy of the substances thus identified in the treatment and prevention of cognitive disorders is confirmed with the aid of known standard animal models for leaming and memory (cf., for example, ‘Alzheimer’s Disease: Biology, Diagnosis and Therapeutics', Iqbal et al., ed.; 1997, John Wiley, pp. 781-786). Suitable animal models for this are, for example, passive or active avoidance behaviour, classical or operant conditioning, spatial orientation tests, or object or subject recognition tests. A particularly suitable model recommended is the ‘Morris test’, which is based on spatial memory (J. Neurosci. Methods 1984, 11, 47-60).

4. Morris Test

Spatial orientation learning is recorded in rodents using the Morris test. The test is outstandingly suitable for the assessment of the learning- and memory-promoting action of substances. In this test, rats or mice are trained to locate a platform which is invisible to them as the only possibility of escape from a water-filled swimming pool. A method which has proved suitable is to train the animals four times per day over a period of time of 5 days. In the course of this, the test substances are administered on each day of the experiment at a defined time, e.g. 30 min before the first swimming test per day. Controls receive the corresponding vehicle. The learning power of the animals expresses itself in a training-related shortening of the distance swum between the starting position and platform, and also in a reduction of the swimming time until reaching the platform, i.e. the better the animal remembers the location of the platform, the shorter the distance covered and the faster the platform is reached. The test is carried out using cognitively impaired animals, such as old animals or animals having experimentally induced brain damage. Treatment of rats with scopolamine leads to a severe impairment of the learning power in the Morris test. This cognitive deficit is an animal model for Alzheimer's disease.

5. Object Recognition Test

The object recognition test is a memory test. It measures the ability of rats (and mice) to differentiate between known and unknown objects.

The test is carried out as described (Blokland et al. NeuroReport 1998, 9, 4205-4208; Ennaceur, A., Delacour, J., Behav. Brain Res. 1988, 31, 47-59; Ennaceur, A., Meliani, K., Psychopharmacology 1992, 109, 321-330; Prickaerts, et al. Eur. J. Pharmacol. 1997, 337, 125-136).

In a first run, a rat in an otherwise empty relatively large observation arena is confronted with two identical objects. The rat will extensively examine, i.e. sniff and touch, both objects. In a second run, after an interval of 24 hours, the rat is again placed in the observation arena. One of the known objects is now replaced by a new, unknown object. When a rat recognises the known object, it will especially examine the unknown object. After 24 hours, however, the rat has normally forgotten which object it has already examined in the first run, and will therefore inspect both objects equally intensively. The administration of a substance having learning- and memory-improving action will lead to a rat recognizing as known the object already seen 24 hours beforehand, in the first run. It will examine the new, unknown object in greater detail than the already known one. This memory power is expressed in a discrimination index. A discrimination index of zero means that the rat examines both objects, the old one and the new one, for the same length of time; i.e. it has not recognized the old object and reacts to both objects as if they were both unknown and new. A discrimination index of greater than zero means that the rat inspects the new object for longer than the old one; i.e. the rat has recognized the old object.

The new active compounds can be converted in a known manner into the customary formulations, such as tablets, coated tablets, pills, granules, aerosols, syrups, emulsions, suspensions and solutions, using inert, non-toxic, pharmaceutically suitable vehicles or solvents. In this connection, the therapeutically active compound should in each case be present in a concentration of approximately 0.5 to 90% by weight of the total mixture, i.e. in amounts which are sufficient in order to achieve the dosage range indicated.

The formulations are prepared, for example, by extending the active compounds using solvents and/or vehicles, if appropriate using emulsifying agents and/or dispersing agents, where, for example, in the case of the use of water as a diluent, it is optionally possible to use organic solvents as auxiliary solvents.

Administration is carried out in the customary manner, preferably orally, parenterally or topically, in particular perlingually, intravenously or intravitally, if appropriate as a depot in an implant.

In the case of parenteral administration, solutions of the active compounds using suitable liquid carrier materials can be employed.

In general, it has proved advantageous in the case of intravenous administration to administer amounts of approximately 0.001 to 10 mg/kg, preferably approximately 0.01 to 5 mg/kg, of body weight to achieve efficacious results, and in the case of oral administration the dose is approximately 0.01 to 25 mg/kg, preferably 0.1 to 10 mg/kg, of body weight.

In spite of this, if appropriate it may be necessary to deviate from the amounts mentioned, namely depending on the body weight or the type of administration route, on the individual behaviour towards the medicament, the manner of its formulation and the time or interval at which administration takes place. Thus, in some cases it may be adequate to manage with less than the minimum amount previously mentioned, while in other cases the upper limit mentioned must be exceeded. In the case of the administration of relatively large amounts, it may be advisable to divide these into a number of individual doses over the course of the day.

If appropriate, it can be useful to combine the compounds according to the invention with other active compounds.

### EXAMPLES

Starting Compounds

A) Sulphonyl Chlorides

N-Acetyl-indoline-5-sulphonyl chloride was prepared according to a literature process (Borrer et al. J. Org. Chem. 1988, 53, 2047) just as N-acetyl-indoline-6-sulphonyl chloride (Carlier et al. J. Org. Chem. 1994, 59, 3232). N-Acetyl-isoindoline-5-silphonyl chloride was prepared from N-acetylisoindoline and chlorosulphonic acid in analogy to N-acetyl-indoline-5-sulphonyl chloride.

B) Anilines

The necessary anilines were prepared according to literature processes (U.S. Pat. No. 3,979,202; Rajappa et al. J. Chem. Res. Miniprint 1986, 5, 1657; WO 96/31462, Hartmann et al. Eur. J. Med. Chem. Chim. Ther. 1994, 29, 807) as shown in the above schemes.

### Examples

A) N-Acetyl-(iso)indoline-sulphonamides

### Example 26

N-(N-Acetylindoline-5-sulphonyl)-N′-(3-fluoro-2,2-dimethylpropanoyl)-1,3-diaminobenzene

A solution of 3.50 g (16.65 mmol) of N-3-fluoro-2,2-dimethylpropanoyl)-1,3-diaminobenzene in 10 ml of dry THF was added dropwise to a solution of 3.93 g (15.13 mmol) of N-acetyl-indoline-5-sulphonyl chloride and 5.99 g (75.67 mmol) of pyridine in 50 ml of dry THF at 0° C. The ice bath was removed, and the stirring was continued for 24 hours at room temperature. The solvent and excess pyridine were then removed in vacuo. The resulting suspension was treated with a mixture of 25 ml of ether, 5 ml of ethyl acetate and 2-molar aqueous hydrochloric acid. The crystalline product was isolated and washed successively with water and ether. 4.90 g (11.30 mmol), 75% yield of a slightly pale pink-coloured solid were obtained.

R_{f}: 0.40 (ethyl acetate).

¹H-NMR (300 MHz, DMSO-d₆, δ/ppm): 10.09 (1H, s), 9.31 (1H, s), 8.07 (1H, d), 7.62 (1H, s), 7.61 (1H, d), 7.53 (1H, t), 7.24 (1H, dd), 7.11 (1H, t), 6.78 (1H, dd), 4.49 (2H, d), 4.11-(2H, t), 3.13 (2H, t), 2.13 (3H, s), 1.22 (6H, s). MS (ESI+, CH₃CN/H₂O/CH₃CO₂H, m/z): 434.2 (M+H⁺).

The other N-acetyl-indoline- and isoindoline-sulphonamides of Examples 25, 28, 32, 33, 18, 36, 56, 50, 49, 63, 61 and 59 were prepared in the same manner as in the table shown below.

B) N-Ethyl(iso)indoline-sulphonamides

### Example 19

N-(N-Ethylindoline-5-sulphonyl)-N′-(3-fluoro-2,2-dimethylpropanoyl)-1,3-diaminobenzene

A solution of 1.0 g (2.31 mmol) of N-(N-acetylindoline-5-sulphonyl)-N′-(3-fluoro-2,2-dimethylpropanoyl)-1,3-diaminobenzene in 30 ml of dry THF was reacted at 0° C. with 2.80 ml (2.77 mmol) of a 1-molar solution of lithium aluminium hydride in THF. The ice-water bath was removed and the stirring was continued for 2 hours at room temperature. The reaction was ended by addition of methanol. The mixture was diluted with ethyl acetate and washed successively with aqueous potassium sodium tartrate solution, aqueous (5% strength) sodium hydrogenphosphate solution, water and sodium chloride solution and dried over anhydrous sodium sulphate. The product was purified by preparative HPLC. 105 mg (0.25 mmol, 11% yield) of a white solid were obtained.

R_{f}: 0.17 (cyclohexane/ethyl acetate, 1:1).

¹H-NMR (300 MHz, DMSO-d₆, δ/ppm): 9.87 (1H, s), 9.31 (1H, s), 7.51 (1H, t), 7.42 (1H, dd), 7.34 (1H, d), 7.23 (1H, dd), 7.10 (1H, t), 6.78 (1H, d), 4.49 (2H, d), 3.44 (2H, t), 3.17 (2H, quart.), 2.91 (2H, t), 1.05 (3H, t). MS (ESI+, CH₃CN/H₂O/CH₃CO₂H, m/z): 442 (M+Na⁺), 420 (M+H⁺).

In the same manner, the other N-ethylindoline- and -isoindolinesulphonamides of Examples 15, 20, 35, 30, 34, 8, 11, 27, 46, 41, 40, 62, 60 and 57 of the following table were obtained.

C) (Iso)indoline-sulphonamides

### Example 16

4-[N-(5-Indolinesulphonyl]amino-N-(tert-butyl)benzamide

300 mg (0.72 mmol) of [N-(N-acetylindoline-5-sulphonyl)]amino-N-tert-butyl)-benzamide were dissolved in 21 ml of aqueous (5% strength) lithium hydroxide solution. The mixture was kept at 60° C. for 24 hours. After cooling, aqueous (5% strength) sodium hydrogenphosphate solution was added. The aqueous layer was extracted with ethyl acetate. The combined organic extracts were washes successively with water and sodium chloride solution and dried over anhydrous sodium sulphate. The product was recrystallized from ether. 230 mg (0.62 mmol, yield 85%) of a white solid were obtained.

R_{f}: 0.56 (ethyl acetate).

¹H-NMR (400 MHz, DMSO-d₆, δ/ppm): 10.18 (1H, s), 7.63 (2H, d), 7.53 (1H, s), 7.36 (1H, d), 7.35 (1H, s), 7.08 (2H, d), 6.42 (1H, s), 6.41 (1H, d), 3.49 (2H, t), 2.93 (2H, t), 1.32 (9H, s). MS (CI, NH₃, m/z): 391 (M+NH₄⁺), 374 (M+H⁺).

In the same manner, the other indoline and isoindolinesulphonamides of Examples 24, 38, 23, 37, 31, 55, 54, 53, 58, 65 and 64 of the following table were obtained.

D) N-Acetylindole-sulphonamides

### Example 2

4-[N-Acetylindole-5-sulphonyl]amino-N-(tert-butyl)benzamide A mixture of 200 mg (0.481 mmol) of 4-[N-acetylindoline-5-sulphonyl]amino-N-tert-butyl)benzamide and 164 mg (0.722 mmol) of DDQ in 10 ml of dry 1,4-dioxane was heated under reflux for 48 hours. After 6 and 24 hours, 80 mg (0.352 mmol) of DDQ were in each case added. The product was isolated by flash chromatography (silica gel, ethyl acetate/cyclohexane, 4:1). 85 mg (0.206 mmol, 42% yield) of a white solid were obtained.

R_{f}: 0.62 (ethyl acetate).

¹H-NMR (400 MHz, DMSO-d₆, δ/ppm): 10.54 (1H, s), 8.43 (1H, d), 8.11 (1H, d), 8.01 (1H, d), 7.74 (1H, dd), 7.61 (2H, d), 7.53 (1H, s), 7.11 (2H, d), 6.88 (1H, d), 2.66 (3H, s), 1.30 (9H, s). MS (CI, NH₃, m/z): 431 (M+NH₄⁺), 414 (M+H⁺).

In the same manner, the other N-acetylindolesulphonamides of Examples 4, 7, 21, 29, 13, 43, 47 and 48 of the following table were obtained.

E) N-Ethylindolesulphonamides

### Example 1

[lacuna] N-[(N-Ethylindoline)-5-sulphonyl]-N′-(3-fluoro-2,2-dimethylpropanoyl)-1,3-di-aminobenzene and 27 mg (0.25 mmol) of DDQ in 7 ml of dry 1,4-dioxane were heated under reflux for 3 hours. After evaporating the solvent, the product was isolated by flash chromatography (silica gel, cyclohexane/ethyl acetate, 1:1). 55 mg (0.132 mmol, 79% yield) of a white solid were obtained.

R_{f}: 0.30 (cyclohexane/ethyl acetate, 1:1).

¹H-NMR (300 MHz, DMSO-d₆, δ/ppm): 10.08 (1H, s), 9.80 (1H, s), 8.07 (1H, d), 7.63-7.52 (4H, m), 7.21 (1H, d), 7.08 (1H, t), 6.81 (1H, d), 6.59 (1H, d), 4.48 (2H, d), 4.22 (2H, quart.), 1.32 (3H, t), 1.20 (6H, s). MS (CI, NH₃, m/z): 435 (M+NH₄⁺), 418 (M+H⁺).

In the same manner, the other N-ethylindolesulphonamides of Examples 9, 22, 3, 5, 10, 44, 51 and 45 of the following table were obtained.

F) Indolesulphonamides

### Example 6

4-[N-(Indol-5-sulphonyl)]amino-N-(tert-butyl)benzamide

A mixture of 230 mg of 4-(indoline-5-sulphonyl)amino-N-(tert-butyl)benzamide and 210 mg (0.924 mmol) of DDQ in 10 ml of dry 1,4-dioxane was treated under reflux for 4 hours. After evaporating the solvent, the product was isolated by flash chromatography (silica gel, cyclohexane/ethyl acetate, 1:1). 78 mg (0.21 mmol, 34% yield) of a white solid were obtained.

R_{f}: 0.30 (dichloromethane/methanol, 100:5).

¹H-NMR (400 MHz, DMSO-d₆, δ/ppm): 10.58 (1H, s), 10.40 (1H, s), 8.08 (1H, s), 7.60 (2H, d), 7.52-7.50 (4H, m), 7.12 (2H, d), 6.60 (1H, d), 1.30 (9H, s). MS (CI, NH₃, m/z): 743 (2M+H⁺), 389 (M+NH₄⁺), 372 (M+H⁺).

In the same manner, the other indolesulphonamides of Examples 12, 17, 42, 52 and 39 of the following table were obtained.

The following table shows Examples 1 to 65, their structural formulae and the Rf values obtained.

TABLE

| | | Rf |
|---|---|---|
| No. | Compound | value [a] |
| 1 | 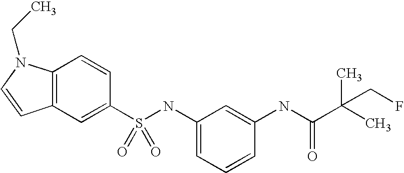 | 0.30 (B) |
| 2 | 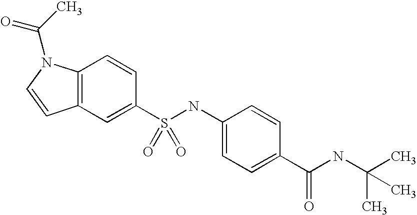 | 0.62 (A) |
| 3 | 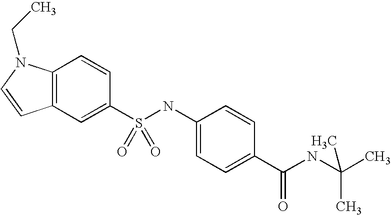 | 0.63 (A) |
| 4 | 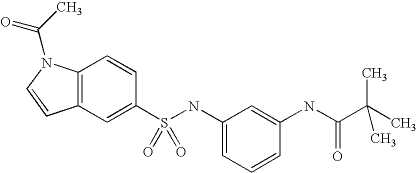 | 0.63 (A) |
| 5 | 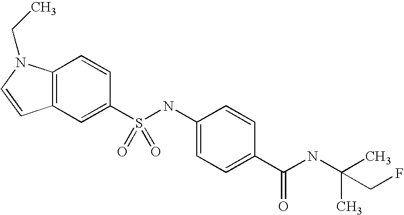 | 0.26 (B) |
| 6 | 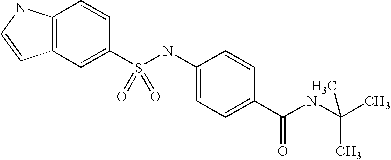 | 0.30 (D) |
| 7 | 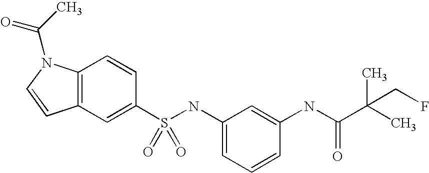 | 0.66 (A) |
| 8 | 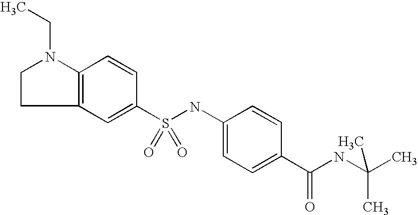 | 0.63 (A) |
| 9 | 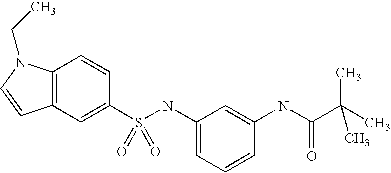 | 0.38 (B) |
| 10 | 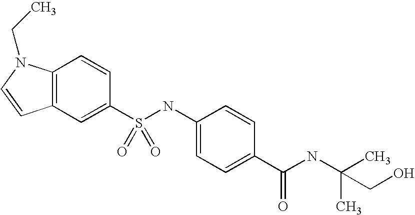 | 0.05 (B) |
| 11 | 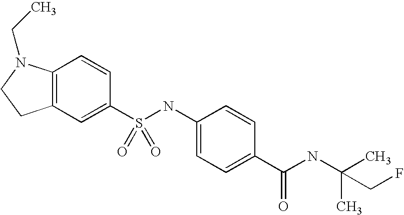 | 0.33 (B) |
| 12 | 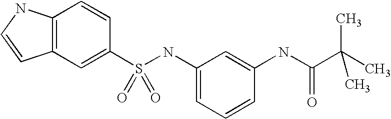 | 0.24 (D) |
| 13 | 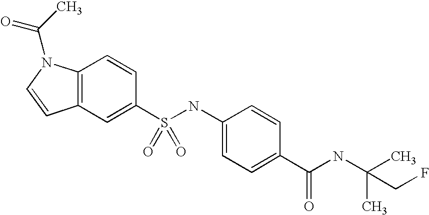 | 0.67 (A) |
| 14 | 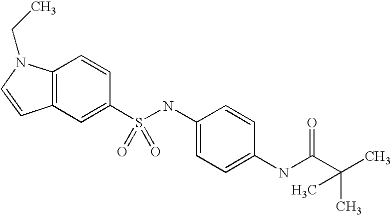 | 0.34 (B) |
| 15 | 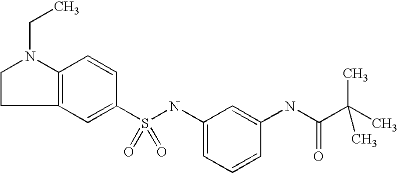 | 0.12 (C) |
| 16 | 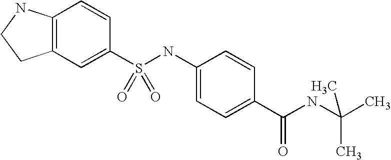 | 0.56 (A) |
| 17 | 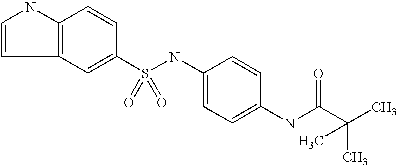 | 0.23 (D) |
| 18 | 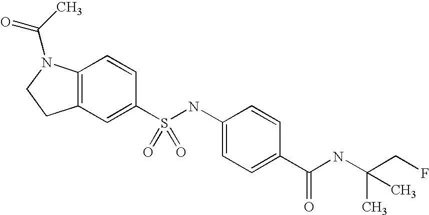 | 0.35 (A) |
| 19 | 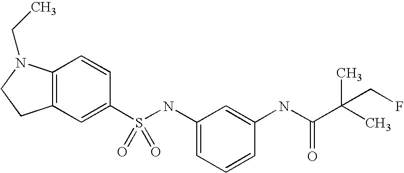 | 0.17 (B) |
| 20 | 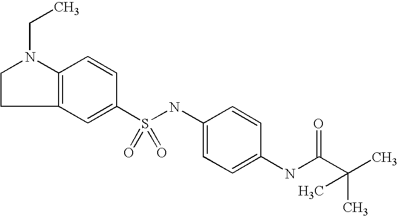 | 0.66 (D) |
| 21 | 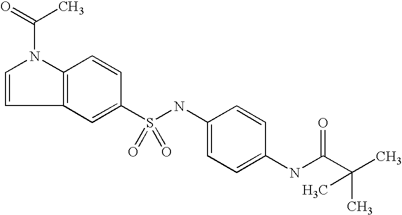 | 0.34 (D) |
| 22 | 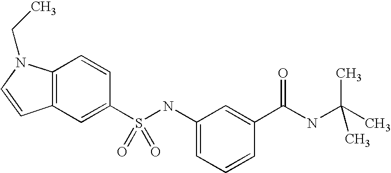 | 0.29 (B) |
| 23 | 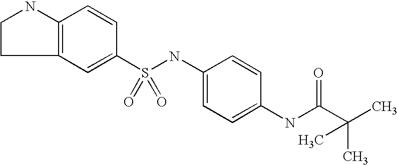 | 0.58 (A) |
| 24 | 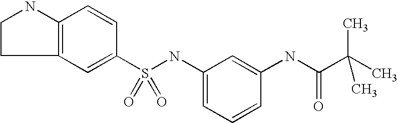 | 0.67 (A) |
| 25 | 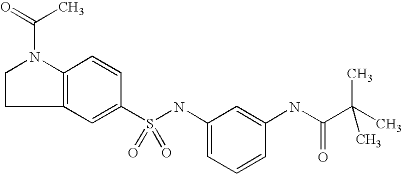 | 0.38 (A) |
| 26 | 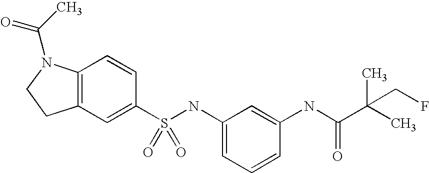 | 0.40 (A) |
| 27 | 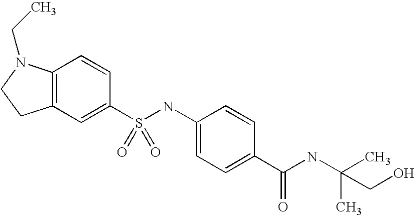 | 0.06 (B) |
| 28 | 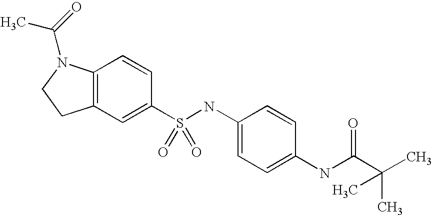 | 0.31 (A) |
| 29 | 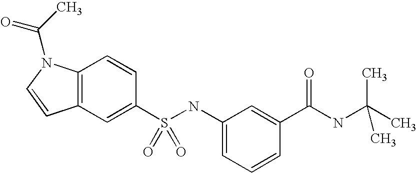 | 0.53 (A) |
| 30 | 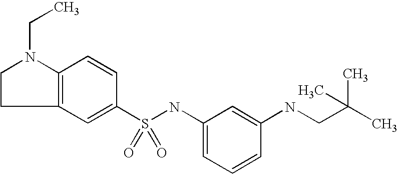 | 0.36 (C) |
| 31 | 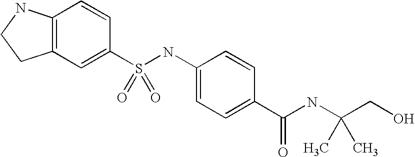 | 0.32 (A) |
| 32 | 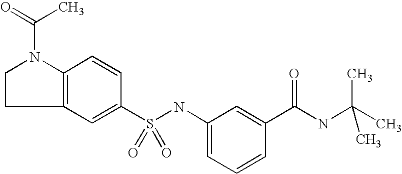 | 0.33 (A) |
| 33 | 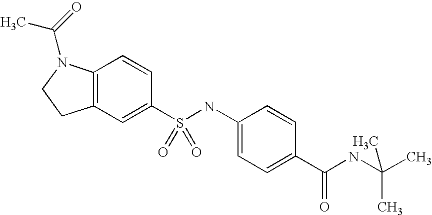 | 0.16 (B) |
| 34 | 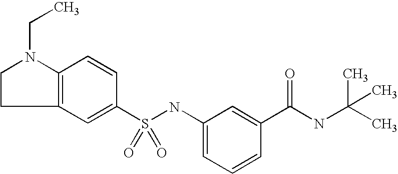 | 0.71 (A) |
| 35 | 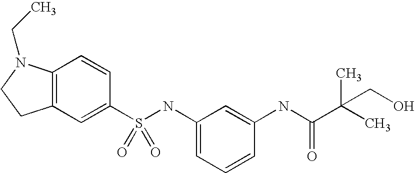 | 0.12 (C) |
| 36 | 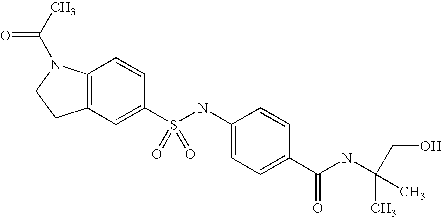 | 0.09 (A) |
| 37 | 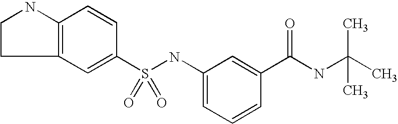 | 0.65 (A) |
| 38 | 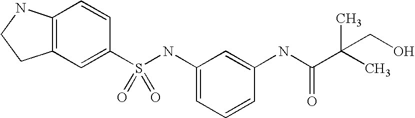 | 0.37 (A) |
| 39 | 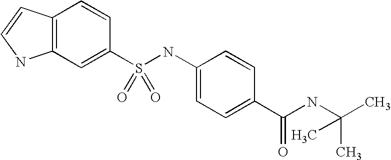 | 0.21 (B) |
| 40 | 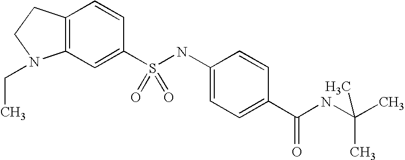 | 0.66 (A) |
| 41 | 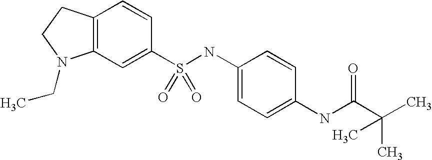 | 0.69 (A) |
| 42 | 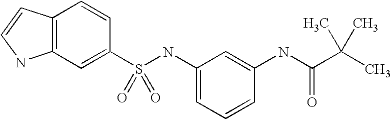 | 0.31 (B) |
| 43 | 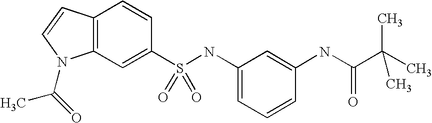 | 0.56 (A) |
| 44 | 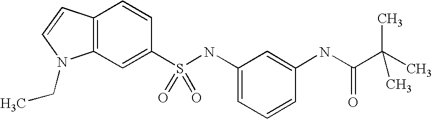 | 0.41 (B) |
| 45 | 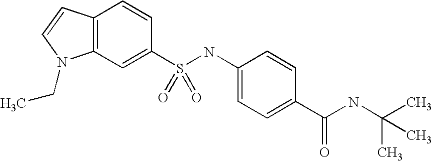 | 0.29 (B) |
| 46 | 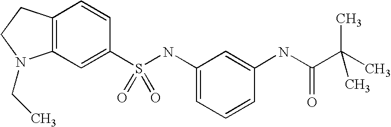 | 0.68 (A) |
| 47 | 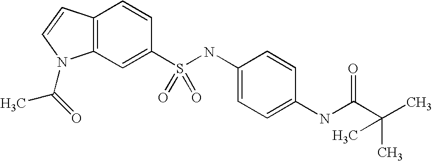 | 0.61 (A) |
| 48 | 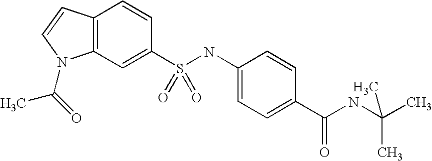 | 0.57 (A) |
| 49 | 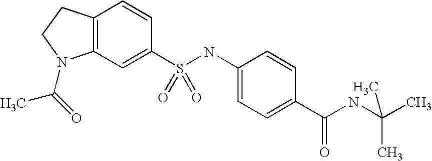 | 0.19 (D) |
| 50 | 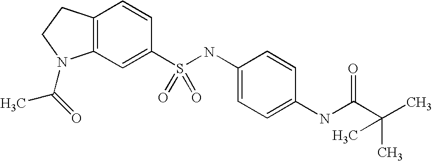 | 0.20 (D) |
| 51 | 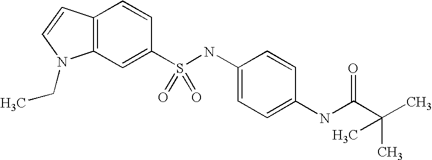 | 0.32 (B) |
| 52 | 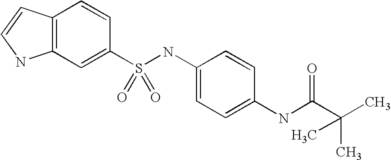 | 0.19 (B) |
| 53 | 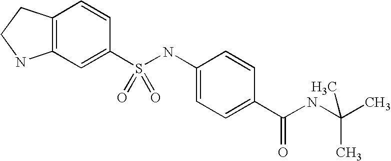 | 0.54 (A) |
| 54 | 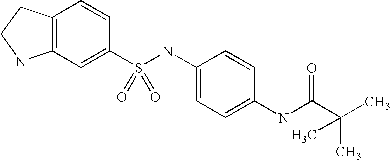 | 0.61 (A) |
| 55 | 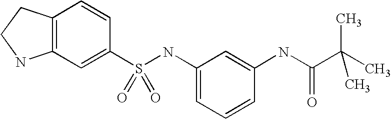 | 0.63 (A) |
| 56 | 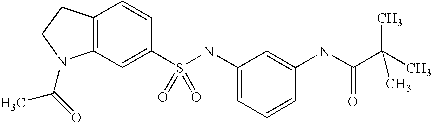 | 0.24 (D) |
| 57 | 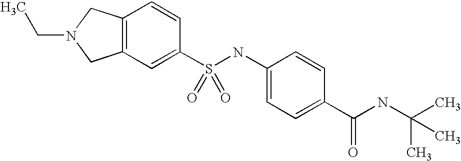 | 0.31 (E) |
| 58 | 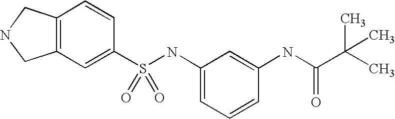 | |
| 59 | 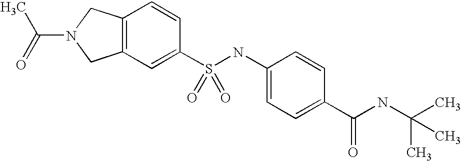 | 0.10 (A) |
| 60 | 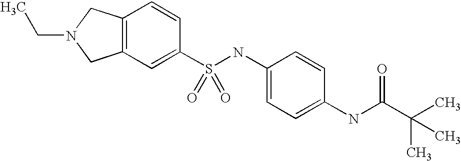 | 0.27 (E) |
| 61 | 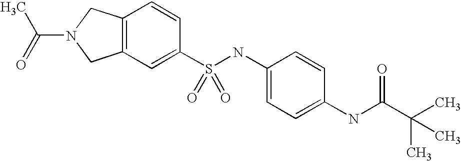 | 0.12 (A) |
| 62 | 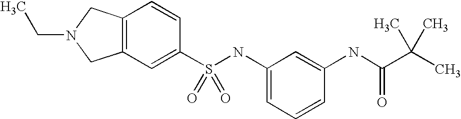 | 0.23 (E) |
| 63 | 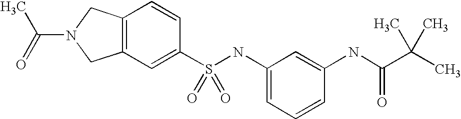 | 0.14 (A) |
| 64 | 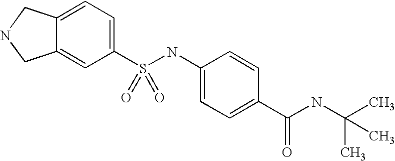 | |
| 65 | 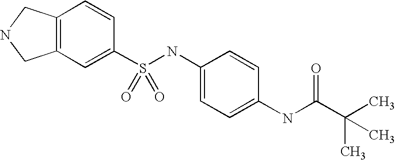 | |

[a]Solvent:
A: Ethyl acetate
B: Cyclohexane/ethyl acetate (v/v = 1:1)
C: Cyclohexane/ethyl acetate (v/v = 2:1)
D: Dichloromethane/methanol (v/v = 100:1)
E: Ethyl acetate/methanol (v/v = 4:1)

He group —N— in the notation of the formulae in the above table means that the group is optionally saturated by a hydrogen atom (—NH—).

1. Use of compounds of the general formula (I) 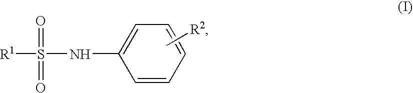
in which
R¹ represents a group which is selected from the following formulae 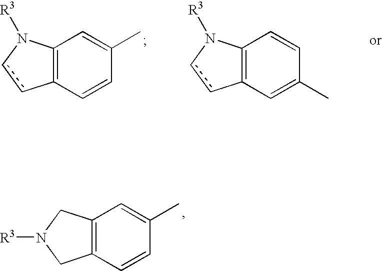
in which [symbol]
represents a single or a double bond,
R³ represents hydrogen, (C₁-C₆)alkyl or (C₃-C₆)cycloalkyl, which in each case can be substituted by 1 to 3 substituents which are selected from the group which consists of hydroxyl, halogen, amino, mono- or di(C₁-C₆)alkylamino, (C₁-C₆)alkanoylamino, (C₁-C₆)alkanoyloxy, (C₁-C₆)alkanoyl, carboxyl, (C₁-C₆)alkoxycarbonyl, carbamoyl, mono- or di(C₁-C₆)alkylaminocarbonyl and cyano, or
R³ represents (C₆-C₁₀)arylsulphonyl, (C₆-C₁₀)arylcarbonyl, whose (C₆-C₁₀)aryl group in each case can be substituted by 1 to 3 substituents which are selected from the group which consists of halogen, (C₁-C₃)alkyl, carboxyl, (C₁-C₃)alkoxycarbonyl, carbamoyl, mono- or di(C₁-C₆)alkylamino-carbonyl, cyano, hydroxyl and (C₁-C₃)alkoxy, or
R³ represents (C₁-C₆)alkanoyl, (C₁-C₆)alkylsulphonyl, (C₃-C₆)cycloalkylcarbonyl, camphorsulphonyl or (C₃-C₆)cycloalkylsulphonyl, or
R³ represents R⁴—X—CO— or R⁴—X—CS—, in which
X represents O, S, NR⁵, in which R⁵ represents hydrogen or (C₁-C₃)alkyl, and.
R⁴ represents (C₁-C₆)alkyl, (C₃-C₆)cycloalkyl, (C₆-C₁₀)aryl or 5-to 10-membered heteroaryl, and
R¹ represents 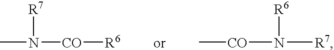
in which
R⁶ is (C₂-C₆)alkenyl or (C₁-C₈)alkyl, which is optionally mono- to trisubstituted identically or differently by amino, protected amino, (C₁-C₄)alkylamino, hydroxyl, cyano, halogen, azido, nitro, trifluoromethyl, carboxyl or phenyl, where phenyl for its part can be substituted up to two times, identically or differently, by nitro, halogen, hydroxyl, (C₁-C₄)alkyl or (C₁-C₄)alkoxy, or
R⁶ represents radicals of the formulae 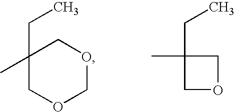
or -L-O—CO-Q
in which
L represents a straight-chain or branched alkanediyl group having up to 6 carbon atoms,
Q represents (C₁-C₆)alkyl, which is optionally substituted by carboxyl, or
represents radicals of the formulae 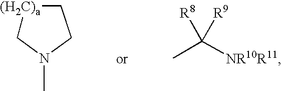
in which
a denotes the number 1 or 2,
R⁸ denotes hydrogen,
R⁹ denotes (C₃-C₈)cycloalkyl, (C₆-C₁₀)aryl or hydrogen, or denotes (C₁-C₈)alkyl,
where the (C₁-C₈)alkyl is optionally substituted by cyano, methylthio, hydroxyl, mercapto, guanidyl or by a group of the formula —NR¹²R¹³ or R¹⁴—OC—,
in which
R¹² and R¹³ independently of one another denote hydrogen, (C₁-C₈)alkyl or phenyl,
and
R¹⁴ denotes hydroxyl, benzyloxy, (C₁-C₆)alkoxy or the abovementioned group —NR¹²R¹³,
or the (C₁-C₈)alkyl is optionally substituted by (C₃-C₈)cycloalkyl or by (C₆-C₁₀)aryl, which for its part is substituted by hydroxyl, halogen, nitro, (C₁-C₈)alkoxy or by the group —NR²R¹³,
in which R¹² and R¹³ have the meaning indicated above,
or the (C₁-C₈)alkyl is optionally substituted by a 5- to 6-membered nitrogen-containing heterocycle or by indolyl, in which the corresponding —NH functions are optionally substituted by (C₁-C₆)allyl or protected by an amino protective group,
R¹⁰ and R¹¹ are identical or different and denote hydrogen or an amino protective group,
R⁷ represents hydrogen or a radical of the formula 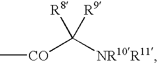
in which
R^{8′}, R^{9′}, R¹⁰′ and R^{11′} have the meaning of R⁸, R⁹, R¹⁰ and R¹¹ indicated above and are identical to or different from this,
and their salts for the production of a medicament for the prophylaxis and/or treatment of diseases which are treatable using a 5-HT₆ receptor antagonist.

2. Use according to claim 1, where the compounds have the general formulae 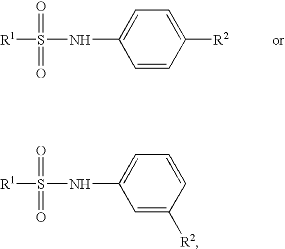
in which R¹ and R² have the meaning indicated in claim 1.

3. Use according to claim 1 or 2, where
R¹ represents a group which is selected from the formulae: 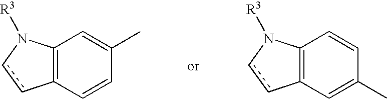
in which [symbol]
represents a single or a double bond, and
R³ has the meaning indicated above.

4. Use according to any one of claims 1 to 3, where
R³ represents hydrogen, (C₁-C₆)alkyl or (C₁-C₆)alkanoyl, and
R² represents 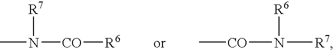
in which
R⁶ is (C₁-C₈)alkyl which is optionally substituted by halogen or hydroxyl, and
R⁷ is hydrogen.

5. Use according to any one of claims 1 to 4, in which R⁶ is tert-butyl which is optionally substituted by halogen or hydroxyl.

6. Use of compounds according to one of claims 1 to 5 for the production of a medicament for the prophylaxis and/or treatment of diseases of the central nervous system.

7. Use according to one of claims 1 to 6, where the disease is a cognitive disorder.

8. Use according to one of claims 1 to 6, where the disease is Alzheimer's disease or another form of dementia.
